SEQUENCE LISTING (5 sequences)

SEQ ID NO: 1 - protein, 7 aa, synthetic construct
VHPKQHR

SEQ ID NO: 2 - protein, 7 aa, synthetic construct
QAHPHVD

SEQ ID NO: 3 - protein, 12 aa, synthetic construct
DITWDQLWDLMK

SEQ ID NO: 4 - protein, 8 aa, synthetic construct
CSRNLIDC

SEQ ID NO: 5 - protein, 8 aa, synthetic construct
CTSDHAVC

(19) United States
(12) Patent Application Publication
(10) Pub. No.: US 2025/0367319 A1
(43) Pub. Date: Dec. 4, 2025

(54) Targeted Nanomedicine for Treating Fibrotic Lung Disorders

(71) Applicant: The University of Chicago, Chicago, IL (US)

(72) Inventors: Yun Fang, Chicago, IL (US); Lauryn Carver, Chicago, IL (US); Matthew Tirrell, Chicago, IL (US); Zhengjie Zhou, Chicago, IL (US)

(21) Appl. No.: 18/875,598

(22) PCT Filed: Jun. 16, 2023
(86) PCT No.: PCT/US2023/068599

§ 371 Date: Dec. 16, 2024

Related U.S. Application Data
(60) Provisional application No. 63/353,464, filed on Jun. 17, 2022.

(51) Int. Cl. A61K 47/69; A61K 9/00; A61K 47/64; A61P 11/00; C12N 15/113

(52) U.S. Cl. CPC A61K 47/6935; A61K 9/0043; A61K 47/644; A61K 47/6939; A61P 11/00; C12N 15/1137; C12N 2310/14; C12N 2310/531; C12N 2320/32; C12Y 503/04001

(57) ABSTRACT

This disclosure relates to compositions and methods for treating lung disorders, including, for example, pulmonary fibrosis, and other fibrotic disorders. Thioredoxin domain-containing 5 (TXNDC5) is significantly increased in fibrotic lungs from human PF patients. Therefore, a targeted nanoparticle comprising an inhibitor of TXNDC5 was developed, which may have potential to treat pulmonary fibrosis.

### CROSS REFERENCE TO RELATED APPLICATIONS

This application claims the benefit of U.S. Provisional Application No. 63/353,464, filed Jun. 17, 2022, which is incorporated by reference herein in its entirety.

### GOVERNMENT SUPPORT

This invention was made with government support under grant numbers R01 HL159558-01A1 and R35 HL161244-01 awarded by the National Institutes of Health. The government has certain rights in the invention.

### REFERENCE TO AN ELECTRONIC SEQUENCE LISTING

The instant application contains an electronic Sequence Listing that has been submitted electronically and is hereby incorporated by reference in its entirety. The sequence listing was created on Jun. 15, 2023, is named “21-0632-WO_SequenceListing.xml” and is 5,443 bytes in size.

### BACKGROUND OF THE DISCLOSURE

### Field of Invention

This disclosure relates to compositions and methods for treating lung disorders, including, for example, pulmonary fibrosis.

### Technical Background

One chronic debilitating consequence of acute respiratory distress syndrome (ARDS) is pulmonary fibrosis (PF) Matthay et al. (J. Clin. Invest. 2012, 122(8), 2731-2740). PF is a common sequela seen in survivors of the acute phase of ARDS with substantial morbidity and mortality (commonly called fibroproliferative ARDS, during which lung injury transitions to aberrant healing) as shown by Meduri et al. (Chest. 1995, 107(4), 1062-1073). PF leads to distorted pulmonary architecture, impaired breathing mechanics and alveolar gas exchange, resulting in hypoxemia, dyspnea, exercise intolerance and ultimately, early death. Importantly, the number of patients who accrue PF increases with time in ARDS, with up to (61%) of patients with a disease duration of greater than 3 weeks. PF is associated with high mortality and/or prolonged ventilator dependence in around 25% of ARDS survivors at 180 days. PF is also associated with a substantially reduced health-related quality of life that can last for months to years. Survivors of pandemic influenza have an increased risk of developing pulmonary fibrosis. Similarly, COVID-19 patients with ARDS develop PF—they have high rates of impaired gas transfer (57%) and reduced lung capacity (25%), both of which increase with disease severity. The COVID-19 pandemic could include a substantial cohort of affected patients with PF and consequent physical impairment.

PF is induced due to abnormal wound healing after inflammatory lung injury. The principal mechanism of PF is activation of lung fibroblasts in a Transformation Growth Factor Beta (TGFβ)-dependent manner with abnormal deposition of mesenchyme and has been demonstrated by Fernandez et al. (Proc. Am. Thorac. Soc. 2012, 9(3), 111-116). Activations of fibrogenic pathways accompany viral infection. Despite scientific understanding and therapeutic advancement, mortality reduction by current anti-fibrotic regimens has only been shown using pooled data from multiple clinical trials exampled by Richeldi et al. (Res. Med. 2016, 113, 74-79), and has only begun to address non-IPF (idiopathic pulmonary fibrosis) pulmonary fibrosis as in Flaherty et al. (N. Engl. J. Med., 2019, 381(18), 1718-1727). Definitive treatment is lung transplant which is fraught with its own complications. With the COVID-19 ARDS population, there is an unmet need for rapid advancement in PF therapeutics shown in George et al. (Lancet Respir. Med. 2020, 8(8), 807-815).

Thioredoxin-domain containing-5 (TXNDC5) is a protein disulfide isomerase enriched in the endoplasmic reticulum. Recently, a novel role of fibroblast TXNDC5 in fibroblast activation and pulmonary fibrosis was described by Lee et al. (Nat. Commun., 2020, 11(1), 4254). The results showed that TXNDC5 is significantly increased in fibrotic lungs from human PF patients and from mice treated with bleomycin (a well-established method to induce mouse PF). A new molecular mechanism was delineated demonstrating PF-associated TXNDC5 is enriched in fibroblasts and TXNDC5 stabilizes transforming growth factor beta receptor I (TGFβR1) to activate fibroblasts during lung fibrosis (see FIGS. 1A-1E).

A recent study demonstrated a causal role of fibroblast TXNDC5 in driving pulmonary fibrosis in vivo by Lee et al. (Nat. Commun., 2020, 11(1), 4254). A new mouse line was engineered in which TXNDC5 was specifically deleted by a Cre-recombinase driven by a Col1a2 (Collagen, type I, alpha 2) promoter restricted to fibroblasts. Pulmonary fibrosis was induced in mouse lungs by intratracheal delivery of bleomycin which damages the lung tissues and stimulates fibroblasts. Results demonstrated that fibroblast specific knockout of Txndc5 in a Col1a2-cre/ERT2*Txndc5^{fl/fl} (Txndc5^{cKO}) mouse significantly reduced the fibrotic area induced by bleomycin (BLM), as shown by collagen content quantified by Picrosirius Red (FIG. 1C), second harmonic generation (FIG. 1D), and hydroxyproline content (FIG. 1E). Collectively, these data demonstrated that TXNDC5 is induced during lung injury and is necessary for fibrotic progression.

Currently, available therapeutic options for PF remain suboptimal, underscoring unmet medical needs in a heightened state due to the COVID-19 pandemic. PF is underserved by the nanomedicine community. Strongly supported by previously published and unpublished in vivo results, it is believed that targeted nanomedicine approaches have tremendous potential to treat pulmonary fibrosis. Therefore, targeted nanomedicine approaches with tremendous potential to treat pulmonary fibrosis were developed as described herein.

### SUMMARY OF THE DISCLOSURE

This disclosure describes compositions and methods for treating lung disorders, including, for example, pulmonary fibrosis (PF), and other fibrotic disorders.

In a first aspect, the present disclosure provides a targeted nanoparticle, comprising an inhibitor of thioredoxin domain-containing 5 (TXNDC5). In some embodiments of the first aspect, the targeted nanoparticle comprises a polyethylene glycol (PEG) domain, and a Platelet Derived Growth Factor Receptor Beta (PDGFRB) targeting molecule. In some embodiments, the targeted nanoparticle further comprises poly-L-arginine, hyaluronic acid (HA), and/or a fluorinated polyethylenimine (PEI). In some embodiments, the PDGFRB targeting molecule comprises a peptide comprising the amino acid sequence CSRNLIDC (SEQ ID NO: 4), a peptide described by Beljaars et al. (Biochem. Pharmacol. 2003, 66(7), 1307-1317). In some embodiments, the targeted nanoparticle comprises CSRNLIDC (SEQ ID NO: 4), the PEG domain, and poly-L-arginine. In some embodiments, the PDGFRB targeted nanoparticle comprises (HA)-PEG-PDGFRB targeting peptide, and/or a fluorinated polyethylenimine (PEI). In some embodiments, the inhibitor of TXNDC5 is a short hairpin RNA (shRNA) silencing TXNDC5 (shTXNDC5), or a TXNDC5-targeting CRISPR plasmid. In some embodiments, the TXNDC5 inhibitor comprises a concentration of about 2 μM or more. In some embodiments, the PEG domain comprises PEG having an average molecular weight of about 1,000 to about 100,000 Daltons.

In a second aspect, the present disclosure provides a pharmaceutical composition, comprising a therapeutically effective amount of the targeted nanoparticle comprising an inhibitor of TXNDC5 as disclosed herein, and a pharmaceutically acceptable carrier, solvent, adjuvant, and/or diluent. In some embodiments, the pharmaceutical composition is formulated for oral, intravenous, topical, ocular, buccal, systemic, nasal, injection, transdermal, rectal, or vaginal administration. In some embodiments, the pharmaceutical composition is formulated for inhalation or insufflation.

In a third aspect, the present disclosure provides a pharmaceutical composition for pulmonary delivery of an inhibitor of TXNDC5 comprising a targeted nanoparticle comprising a PDGFRB targeting molecule, a polyethylene glycol (PEG) domain, and the inhibitor of TXNDC5; and a pharmaceutically acceptable carrier, wherein the composition is formulated such that once administered to the lung, it results in the delivery of the inhibitor of TXNDC5 to a lung cell. In some embodiments, the pharmaceutical composition further comprises poly-L-arginine, hyaluronic acid (HA), and/or a fluorinated polyethylenimine (PEI). In some embodiments, the pharmaceutical composition comprises a PDGFRB targeting molecule comprising the amino acid sequence CSRNLIDC (SEQ ID NO: 4). In some embodiments, the pharmaceutical composition comprises a PDGFRB targeted nanoparticle comprising CSRNLIDC (SEQ ID NO: 4), the PEG domain, and poly-L-arginine. In some embodiments, the pharmaceutical composition comprises a PDGFRB targeted nanoparticle comprising CSRNLIDC (SEQ ID NO: 4), the PEG domain, HA, and fluorinated polyethylenimine (PEI). In some embodiments, the pharmaceutical composition comprises an inhibitor of TXNDC5 that is a short hairpin RNA (shRNA) silencing TXNDC5 (shTXNDC5), a TXNDC5-targeting small interfering (siRNA), or a TXNDC5-targeting CRISPR plasmid.

In a fourth aspect, the present disclosure provides a method of treating a lung disorder in a subject, comprising administering to the subject a therapeutically effective amount of a pharmaceutical composition as described herein comprising a targeted nanoparticle comprising an inhibitor of TXNDC5, wherein the targeted nanoparticle is preferentially targeted to fibrotic fibroblast cells associated with the lung disorder; and reducing fibrosis at the site of the fibrotic fibroblast. In some embodiments of the fourth aspect, the lung disorder comprises pulmonary fibrosis (PF). In some embodiments of the fourth aspect, the method results in one or more of an inhibition of TXNDC5 and reduction in fibrosis compared to a control at the site of the fibrotic fibroblast cells. In some embodiments of the fourth aspect, the pulmonary fibrosis is an idiopathic pulmonary fibrosis.

In a fifth aspect, the present disclosure provides a method of promoting fibroblast wound healing in a subject comprising administering to the subject a therapeutically effective amount of a pharmaceutical composition as described herein comprising a targeted nanoparticle comprising an activator or an inhibitor of TXNDC5, wherein the targeted nanoparticle is preferentially targeted to fibrotic fibroblast cells associated with the fibroblast wound. In some embodiments of the fifth aspect, the probability of survival of the individual is at least about 10% greater than an expected probability of survival without administration of the pharmaceutical composition.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIGS. 1A-1E. Fibroblast TXNDC5 drives pulmonary fibrosis. (FIG. 1A) Increased TXNDC5 protein in fibrotic human and mouse lungs. Error bars represent SEM. (FIG. 1B) Schematic summary describing the pro-fibrotic role of TXNDC5 in activated fibroblasts. Significantly reduced lung fibrosis in mice in which TXNDC5 is specifically deleted in fibroblasts, demonstrated by (FIG. 1C) Picrosirius Red staining, (FIG. 1D) Second Harmonic Generation, and (FIG. 1E) Hydroxyproline, three assays which quantify collagen. Data are Mean±SEM.

FIGS. 2A-2B. (FIG. 2A) Formulation of PDGFRB-targeting nanoparticles (NPs) encapsulating plasmids expressing shTXNDC5. (FIG. 2B) Reduced TXNDC5 in mouse fibroblasts treated with PDGFRB-targeting PEI NP delivering shTXNDC5 plasmids when compared to eGFP plasmids. (n=7). Data are mean±SEM.

FIGS. 3A-3C. (FIG. 3A) Example 1 study design. (FIG. 3B) Intranasal administration of PDGFRB-targeting PEI nanoparticles delivers functional eGFP plasmids to activated fibroblasts expressing COL1A1/aSMA and (FIG. 3C) reduces lung fibrosis when carrying shTXNDC5 plasmids. (n=5-6). Data are mean±SEM.

FIG. 4 shows the formulation of the targeting peptide-polyethylene glycol (PEG)-poly(L-arginine)-Plasmid Polyelectrolyte Complex Micelle (PCM).

FIG. 5 shows synthesis of targeting peptide-PEG-poly-L-arginine molecules.

FIG. 6A-6B show micelle size and size dispersity as characterized by dynamic light scattering (DLS) and transmission electron microscopy (TEM). Polyelectrolyte Complex Micelle were prepared in Milli-Q purified water and measured directly. Peptide (CSRNLIDC; SEQ ID NO: 4)-Polyethylene Glycol-Poly-L-Arginine/plasmid Polyelectrolyte Complex Micelles were prepared in 50 μL of Milli-Q purified water (1:2 w/w plasmid to polycation). 100 μL Milli-Q purified water was added after the 20 minutes of complexation time. (FIG. 6A) Dynamic Light Scattering Analysis: Intensity weighted size distribution of the Peptide (CSRNLIDC; SEQ ID NO: 4)-Polyethylene Glycol-Poly-L-Arginine/plasmid Polyelectrolyte Complex Micelles (1:2 w/w plasmid to polycation). (FIG. 6B) Transmission electron micrographs of the Peptide-Polyethylene Glycol-Poly-L-Arginine/plasmid Polyelectrolyte Complex Micelles (1:2 w/w plasmid to polycation) samples. Scale bar represents 100 nm. Samples were prepared for TEM imaging by dropping 3.5 μL of micelle solution onto a glow-discharged carbon-coated copper 200-mesh TEM grids for 1 minute. Excess liquid was wicked away. The grid was then rinsed twice with uranyl formate solution (0.75% wv) and then stained for 45 seconds with a third drop (˜25 μL each). Excess liquid was wicked away from the edge of the grids and allowed to air dry.

FIG. 7 shows PDGFRB-targeting polyethylene-glycol-poly-L-arginine polyelectrolyte complex micelles which encapsulate Txndc5-targeting CRISPR-Cas9 plasmids successfully suppress Txndc5 expression in mouse fibroblasts. (top) Experimental design for the micelle treatment of murine fibroblasts. Mouse fibroblasts were first activated by TGF-β and then treated with the polyelectrolyte complex micelles which encapsulate Txndc5-targeting CRISPR-Cas9 plasmids or control plasmids. The Txndc5-targeting CRISPR-Cas9 vector was a plasmid engineered to express Cas9 driven by a mouse col1a1 promoter that is preferentially activated in fibroblast. The Txndc5-targeting CRISPR-Cas9 vector also contains two U6 promoter-driven single-guide RNAs (sgRNAs) targeting introns 1 and 3 of Txndc5. The control plasmid was engineered to express Cas9 driven by a mouse col1a1 promoter and contains non-targeting guide RNAs. (bottom) Decreased expression of Txndc5 mRNA in mouse fibroblasts treated with PDGFRB-targeting polyelectrolyte complex micelles encapsulating Txndc5-targeting Cas9 plasmids when compared to fibroblasts treated with PDGFRB-targeting polyelectrolyte complex micelles encapsulating control Cas9 plasmids.

### DETAILED DESCRIPTION

Provided herein are compositions and methods for treating lung disorders, including, pulmonary fibrosis, and other fibrotic disorders. As used herein, the term “lung disorder” refers to disorders, diseases, and/or damage to the lungs of an individual.

It is to be understood that the particular aspects of the specification are described herein are not limited to specific embodiments presented, and can vary. It also will be understood that the terminology used herein is for the purpose of describing particular aspects only and, unless specifically defined herein, is not intended to be limiting. Moreover, particular embodiments disclosed herein can be combined with other embodiments disclosed herein, as would be recognized by a skilled person, without limitation.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indictates otherwise.

In some embodiments, percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio or which have otherwise been approved by the United States Food and Drug Administration as being acceptable for use in humans or domestic animals.

“Therapeutically effective amount” or “effective amount” refers to that amount of a therapeutic agent, such as a TXNDC5 inhibitor, which when administered to a subject, is sufficient to effect treatment (e.g., improve symptoms) for a disease or disorder described herein, such as, for example, pulmonary fibrosis, and other fibrotic disorders. The amount of a compound which constitutes a “therapeutically effective amount” or “effective amount” can vary depending on the compound, the disorder and its severity, and the age, weight, sex, and genetic background of the subject to be treated, but can be determined by one of ordinary skill in the art.

“Treating” or “treatment” as used herein refers to the treatment of a disease or disorder described herein, in a subject, preferably a human, and includes inhibiting, relieving, ameliorating, or slowing progression of the disease or disorder or one or more symptoms of the disease or disorder.

“Subject” refers to a warm blooded animal such as a mammal, preferably a human, which is afflicted with, or has the potential to be afflicted with one or more diseases and disorders described herein.

“Pharmaceutical composition” as used herein refers to a composition that includes one or more therapeutic agents disclosed herein, such as a TXNDC5 inhibitor, a pharmaceutically acceptable carrier, a solvent, an adjuvant, and/or a diluent, or any combination thereof.

As used herein, the term “lung disease” can also be described as a “lung disorder” or “lung injury.”

In view of the present disclosure, the methods and compositions described herein can be configured by the person of ordinary skill in the art to meet the desired need. In general, the disclosed materials and methods provide improvements in treating lung disorders as described herein.

### Overview

Disclosed herein is a targeted nanomedicine approach to deliver an inhibitor of TXNDC5 to activated fibroblasts to treat pulmonary fibrosis. In certain embodiments, the inhibitor of TXNDC5 is a short hairpin RNA (shRNA) silencing TXNDC5. In certain embodiments, the inhibitor of TXNDC5 is a TXNDC5-targeting small interfering (siRNA). In certain embodiments, the inhibitor of TXNDC5 is a TXNDC5-targeting CRISPR plasmid. Capitalizing on recent data showing genetic deletion of TXNDC5 in fibroblasts reduces pulmonary fibrosis in mice, nanoparticles which effectively delivers nucleic acids comprising TXNDC5-targeting agents to activated fibroblasts via a targeting peptide against Platelet Derived Growth Factor Receptor Beta (PDGFRB) expressed in activated fibroblasts were engineered.

The significance of the present disclosure includes at least two aspects. First, it provides novel nanomedicine approaches to treat lung disorders with unmet medical need. Second, it integrates targeted nanomedicine and nucleotide-based therapeutics to create a new avenue for the treatment of various lung diseases including pulmonary fibrosis and other fibrotic disorders. This disclosure provides formualtions of PEI or PCM nanoparticles that target activated fibroblasts and simultaneously delivers nucleic acid based inhibitors of TXNDC5 that reduce fibrosis compared to a control at the site of the fibrotic fibroblast cells.

### Short Hairpin RNAs and Nanomedicine

Short hairpin RNAs (shRNAs) are artificial RNA molecules with tight hairpin turns that can be used to silence target gene expression via RNA interference (RNAi). shRNA sequences are typically encoded in a DNA vector or a DNA plasmid that can be introduced into cells via plasmid transfection or viral tranduction. shRNA molecules can be divided into two main categories based on their designs: (1) simple stem-loop; and (2) microRNA-adapted shRNA. A simple stem-loop shRNA is often transcribed under the control of an RNA Polymerase III (Pol III) promoter. The 50-70 nucleotide transcript forms a stem-loop structure consisting of a 19 to 29 bp region of double-strand RNA (the stem) bridged by a region of predominantly single-strand RNA (the loop) and a dinucleotide 3′ overhang. The simple stem-loop shRNA is transcribed in the nucleus and enters the RNAi pathway similar to a pre-microRNA. The longer (>250 nucleotide) microRNA-adapted shRNA is a design that more closely resembles native pri-microRNA molecules and consists of an shRNA stem structure which may include microRNA-like mismatches, bridged by a loop and flanked by 5′ and 3′ endogenous microRNA sequences. The microRNA-adapted shRNA, like the simple stem-loop hairpin, is also transcribed in the nucleus but is thought to enter the RNAi pathway earlier similar to an endogenous pri-microRNA.

In certain embodiments, the inhibitor of TXNDC5 is a TXNDC5-targeting CRISPR plasmid. In general, the term “CRISPR plasmid” refers to a nucleic acid molecule comprising transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR plasmid is derived from a type I, type II, or type III CRISPR system. In some embodiments, one or more elements of a CRISPR plasmid is derived from a particular organism comprising an endogenous CRISPR system, such as Streptococcus pyogenes. In general, a CRISPR plasmid is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex (e.g. at a site to prevent or disrupt expression of TXNDC5). Full complementarity is not necessarily required, provided there is sufficient complementarity to cause hybridization and promote formation of a CRISPR complex.

### Compositions

In some embodiments, pharmaceutical compositions contemplated herein include a therapeutically effective amount of a targeted nanoparticle including one or more inhibitors of fibrosis, such as, for example, a TXNDC5 inhibitor. Such compositions may further include an appropriate pharmaceutically acceptable carrier, solvent, adjuvant, diluent, or any combination thereof. The exact nature of the carrier, solvent, adjuvant, or diluent will depend upon the desired use (e.g., route of administration) for the composition, and may range from being suitable or acceptable for veterinary uses to being suitable or acceptable for human use.

In some embodiments, pharmaceutical compositions contemplated herein include one or more nanoparticles that carry the one or more TXNDC5 inhibitors, for example, inside the nanoparticle, attached to an external surface of the nanoparticle, or both. In some embodiments, the nanoparticles include one or more targeting moeities attached thereto to enable targeted delivery of the nanoparticle to a desired location. For example, the targeting moeity can target the nanoparticle to a site of fibrosis associated with a lung disease or disorder or wound.

Any therapeutic agents are contemplated herein for combatting fibrosis. For example, contemplated agents include inhibitors of TXNDC5, such as siRNAs or shRNAs that inhibit TXNDC5, or CRISPR plasmids that inhibit TXNDC5.

Such compositions optionally include secondary therapeutic agents (possibly also carried on or in contemplated nanoparticles). Examples of such therapeutic agents include nintedanib and pirfenidone.

TXNDC5 inhibitors of the present disclosure can be administered through a variety of routes and in various compositions. For example, pharmaceutical compositions containing TXNDC5 inhibitors can be formulated for oral, intravenous, topical, ocular, buccal, systemic, nasal, injection, transdermal, rectal, or vaginal administration, or formulated in a form suitable for administration by inhalation or insufflation. In some embodiments of the present disclosure, administration is oral, intratracheal, intranasal, or intravenous.

A variety of dosage schedules is contemplated by the present disclosure. For example, a subject can be dosed monthly, every other week, weekly, daily, or multiple times per day. Dosage amounts and dosing frequency can vary based on the dosage form and/or route of administration, and the age, weight, sex, and/or severity of the subject's disease. In some embodiments of the present disclosure, one or more TXNDC5 inhibitors is administered orally, intratracheally, intranasally, or intravenously, and the subject is dosed on a daily basis.

The therapeutic agents (also referred to as “compounds” herein) described herein (e.g., polyelectrolyte micelles, PEI nanoparticles, PDGFRB targeting molecule, DNA, RNA, or TXNDC5 inhibitor), or compositions thereof, will generally be used in an amount effective to achieve the intended result, for example, in an amount effective to provide a therapeutic benefit to subject having the particular disease being treated. As used herein, therapeutic benefit refers to the eradication or amelioration of the underlying disease being treated and/or eradication or amelioration of one or more of the symptoms associated with the underlying disease such that a subject being treated with the therapeutic agent reports an improvement in feeling or condition, notwithstanding that the subject may still be afflicted with the underlying disease.

Determination of an effective dosage of compound(s) for a particular disease and/or mode of administration is well known. Effective dosages can be estimated initially from in vitro activity and metabolism assays. For example, an initial dosage of compound for use in a subject can be formulated to achieve a circulating blood or serum concentration of the metabolite active compound that is at or above an IC₅₀ of the particular compound as measured in an in vitro assay. Calculating dosages to achieve such circulating blood or serum concentrations taking into account the bioavailability of the particular compound via a given route of administration is well within the capabilities of a skilled artisan. Initial dosages of compound can also be estimated from in vivo data, such as from an appropriate animal model.

Dosage amounts of TXNDC5 inhibitors can be in the range of from about 0.0001 mg/kg/day, about 0.001 mg/kg/day, or about 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, including particular condition being treated, the severity of existing or anticipated physiological dysfunction, the genetic profile, age, health, sex, diet, and/or weight of the subject. Dosage amounts and dosing intervals can be adjusted individually to maintain a desired therapeutic effect over time. For example, the compounds may be administered once, or once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

For example, a dosage contemplated herein can include a single volume of about 0.1, 0.2, 0.3, 0.4, 0.5, 0.6, 0.7, 0.8, 0.9, 1.0, 1.5, 2.0, 2.5, or 3.0 mL of a pharmaceutical composition having a concentration of a TXNDC5 inhibitor at about 0.00001, 0001, 0.001, 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 10, 15, 20, 50, 100, 200, 500, or 1000 mM in a pharmaceutically acceptable carrier.

### Nanoparticles

The present disclosure contemplates use of polyelectrolyte complex micelles (PCM) or polyethylenimine (PEI) nanoparticles (also referred to as nanoparticles herein) to deliver therapeutic agents. Polymers that bear charge in an aqueous environment are called polyelectrolytes. When oppositely charged polymers are mixed under the right conditions, they form complexes. Polyelectrolytes, including at least one attached to a non-charged, water soluble block, can be mixed at a stoichiometric charge ratio with an oppositely charged homopolymer to form particles of a relatively compact core surrounded by a dilute corona of neutral water soluble block. These nanometer-sized particles are called polyelectrolyte complex micelles, polyion complex micelles, interpolyelectrolyte complex micelles, complex coacervate core micelles, or polyelectrolyte micelles. Polyelectrolyte complexes composed of nucleic acids and positively charged polymers have been explored as a possibility to neutralize the charge on the molecule and protect it from enzymatic degradation. Polyelectrolyte complex micelles have great potential as gene delivery vehicles because of their ability to encapsulate charged nucleic acids, forming a core by neutralizing their charge, while simultaneously protecting the nucleic acids from non-specific interactions and enzymatic degradation. Furthermore, to enhance specificity and transfection efficiency, polyelectrolyte complex micelles can be modified to include targeting capabilities.

The contemplated polyelectrolyte micelles can comprise polyethylene glycol (PEG) domains. PEG domains prevent macrophase separation, stabilizing the micelles. The domains further protect the nanoparticles from recognition by the reticuloendothelial system in the body. The PEG domain can be comprised of PEG having an average molecular weight of about 1,000 to about 100,000 Daltons (Da). In some embodiments, the contemplated nanoparticle can comprise a polyethylene glycol (PEG)-2000.

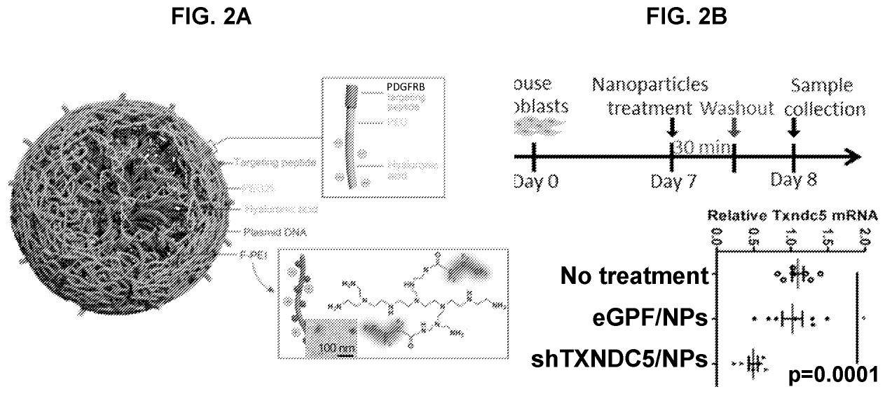

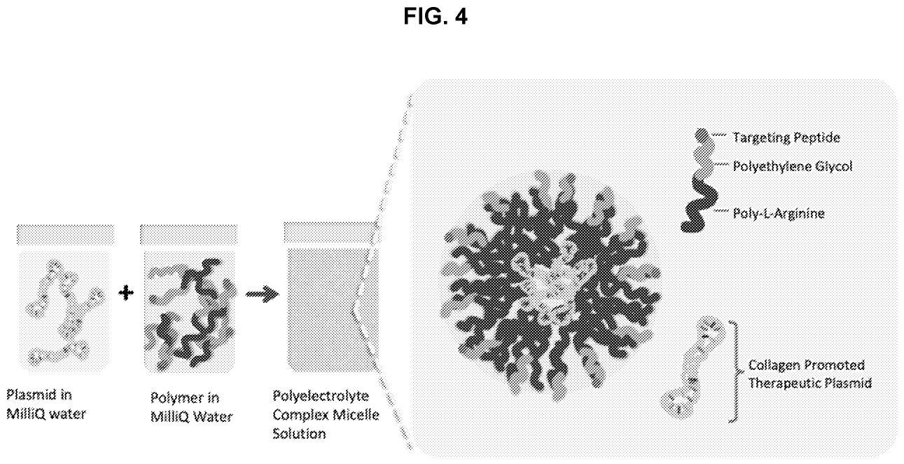

Contemplated nanoparticles for use herein include, for example, polyelectrolyte complex micelles that can effectively incorporate negatively-charged nucleotides in the core and functionally display tissue-targeting peptides on the surface. These self-assembled nano-scale carriers (˜80 nm in diameter (when hydrated)) are formed by electrostatic interaction between two oppositely-charged polymers. Polyethylene glycol (PEG)-2000 conjugated with poly-L-arginine at one end and a Platelet Derived Growth Factor Receptor Beta (PDGFRB) targeting molecule at the other end, and an inhibitor of TXNDC5 (see FIG. 4). In certain embodiments, the contemplated nanoparticle can comprise a Polyethylene glycol (PEG)-2000 conjugated with a hyaluronic acid (HA) and a Platelet Derived Growth Factor Receptor Beta (PDGFRB) targeting molecule, a fluorinated polyethylenimine (PEI), and an inhibitor of TXNDC5 (see FIG. 2). Negatively-charged nucleotides are neutralized by poly-lysine or PEI and encapsulated in the cores of the nanoparticles. This approach offers multiple advantages, including: (i) the nano-scale of micelles significantly increases the surface area: volume ratio that can enhance specific targeting, and (ii) the self-assembling feature of the PCM or PEI nanoparticles eliminates the use of chemical cross-linking agents, thereby reducing possible toxicities.

Additional micelles are contemplated for use herein, such as those disclosed in International Application No. PCT/US2006/020760 (U.S. Pat. No. 9,505,867), Vieregg et al. (J. Am. Chem. Soc. 2018, 140, 1632-1638), Lueckheide et al. (Nano Lett. 2018, 18, 7111-7117), and Marras et al. (Polymers 2019, 11, 83), each of which is incorporated by reference.

### Targeting Molecules

The present disclosure contemplates use of targeting molecules (or targeting moieties) with the nanoparticles disclosed herein for targeted delivery of therapeutic compositions, such as TXNDC5 inhibitors, or for incorporation into pharmaceutical compositions as described herein. Targeting molecules can be PDGFRB-targeting molecules. Targeting molecules can include peptides such as CSRNLIDC (SEQ ID NO: 4), which was and is a cyclic peptide with a disulfide bond between cysteines that binds specifically to PDGFRB. PDGFRB is a membrane receptor. Single-cell profiling and mechanistic studies demonstrated that PDGFRB expression is enriched and significantly increased in activated fibroblasts in fibrotic lungs shown in Xie et al. (Cell Rep., 2018, 22(13), 3625-3640) and Hewitt et al (J. Cell. Sci, 2012, 125(9), 2276-2287).

In one embodiment, a contemplated targeted nanoparticle containing a TXNDC5 inhibitor (2 μM) is (HA)-PEG-PDGFRB targeting peptide. In another embodiment, a contemplated targeted nanoparticle containing a TXNDC5 inhibitor is poly-L-arginine-PEG-PDGFRB targeting peptide.

In some embodiments, contemplated nanoparticles exhibit a polydispersity of about 0.1 to about 0.3.

In some embodiments, contemplated nanoparticles exhibit a spherical shape and have a diameter (in nanometers, nm) of about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, about 70, about 75, about 80, about 85, about 90, about 95, about 100, about 105, about 110, about 115, about 120, or about 125 nm. In some embodiments, the nanoparticles have a radius of about 20 nm to about 80 nm.

The nanoparticle delivery system of the present disclosure possesses multiple advantages compared with other nanoparticle-based platforms. The first advantage is higher stability: the therapeutically active components (e.g., TXNDC5 inhibitors or other nucleic acid based therapeutics) can be encapsulated in the inner core of the polyelectrolyte complex micelles or PEI-based nanoparticle and can therefore be protected by the outer layer of biocompatible polymers. Through this approach, first, the degradation of therapeutic nucleotides contained therein by serum nucleases is prevented; second, the NPs are capable of escaping renal clearance; and third, immunogenic responses are avoided. The second advantage is higher safety: cell-targeting peptides (e.g., those targeting to PDGFRB) are covalently conjugated on the periphery of the polyelectrolyte complex micelles or PEI nanoparticles, which significantly reduces cytotoxicity and increases circulation time by circumventing nonspecific interaction with serum components. The third advantage is higher specificity: with the defined chemical structures of the targeting peptides, the polyelectrolyte complex micelles and PEI nanoparticles are able to bind specific receptors and penetrate targeted cells. A further advantage is higher scalability. This approach does not require chemical modifications on nucleotides for conjugation, nor does it need to engineer hard-to-reproduce lipid nanoparticles. The synthesis of the core components in these nanoparticles is highly automated. In addition, the targeting peptides are easily changeable to target different receptors.

Further, as shown herein, the use of targeted nanoparticles permits use of a lower amount of a therapeutic agent for the treatment of a given disorder or wound due to the specific targeting of the therapeutic agent to the site of the disorder or wound. In this way, use of targeted nanoparticles can significantly lower the dosage of a therapeutic agent required to treat a disorder or wound, which can significantly reduce costs associated with the treatment. For example, a therapeutically effective amount of a therapeutic agent to be delivered by a targeted nanoparticle can be at least about 10, 20, 30, 40, or 50% lower than the therapeutically effective amount of the naked (non-targeted) therapeutic agent.

### Methods

In some embodiments, methods of treating and/or preventing a lung disorder in a subject in need thereof include administering to the subject a therapeutically effective amount of one or more TXNDC5 inhibitors. Treatable and/or preventable lung disorders can include pulmonary fibrosis and associated sequelae.

In some embodiments, therapeutic methods contemplated herein can also treat and/or prevent complications associated with or promote fibroblast wound healing (e.g., caused by lung injury) by administering to the subject a therapeutically effective amount of one or more agents which increase or decrease TXNDC5.

In some embodiments, therapeutic methods contemplated herein can also accelerate fibroblast growth to treat wound healing (e.g., caused by lung injury) by administering to the subject a therapeutically effective amount of one or more agents which increase or decrease TXNDC5.

The present disclosure contemplates methods that result in a variety of indications of improvement for pulmonary fibrosis.

The present disclosure contemplates a variety of methods of administering the therapeutic agents, targeting molecules, and nanoparticles disclosed herein, including local, oral, nasal, tracheal, rectal, intravaginal, topical, subcutaneous, intradermal, intramuscular (IM), intravenous (IV), intrathecal (IT), intracerebral, epidural, or intracranial administration. Local, in situ administration of these compositions is contemplated.

### EXAMPLES

The Examples that follow are illustrative of specific embodiments of the disclosure, and various uses thereof. They are set forth for explanatory purposes only and should not be construed as limiting the scope of the disclosure in any way.

The research approach described here employed state-of-the-art, complementary in vitro, in vivo (mouse) and ex vivo (human) investigations of lung injury and pulmonary fibrosis. Human lung experiments included at least 12 biological replicates (distinct human donors); data reported reflect these biological replicates and not technical replicates. Translational animal experiments were conducted in accordance with the ARRIVE guidelines. Both male and female mice were blindly randomized to experimental or control groups, minimizing subjective bias and controlling for sex effects of disease (where appropriate—fibrosis is regulated by mouse sex). Rigorous contemporaneous controls were used. Animal drop-out (e.g. mortality) are reported. All analyses were performed by observers blinded to treatment allocation. All assays were performed in a blinded manner in triplicate. Statistical outliers were explicitly reported. In vivo animal studies include the experimental treatment with nanoparticles to determine effectiveness for decreasing PF (Examples 1 and 2). Using Stata software version 6, a power of analysis calculation from previous studies, an estimated minimum of 25 mice per condition were required to reach significance for analyses. A senior biostatistician was involved with all aspects of study design, data collection, and analysis.

### Targeted Nanomedicine Approaches Treating Pulmonary Fibrosis

Recent data provide a mechanistic basis for targeting fibroblast TXNDC5 to treat pulmonary fibrosis; nevertheless, the challenge was to develop an effective strategy to deliver therapeutics inhibiting TXNDC5 in activated fibroblasts. To overcome this, a nanoparticle that effectively delivers shTXNDC5-expressing plasmids to activated fibroblasts via a targeting peptide against Platelet Derived Growth Factor Receptor Beta (PDGFRB) was engineered and optimized. PDGFRB is a membrane receptor. Single-cell profiling and mechanistic studies demonstrated that PDGFRB expression is enriched and significantly increased in activated fibroblasts in fibrotic lungs. A cyclic peptide (CSRNLIDC (SEQ ID NO: 4) with a disulfide bond between cysteines) was identified to bind specifically to PDGFRB. This peptide was displayed on the nanoparticle surface to target PDGFRB-expressing fibroblasts. Fluorinated polyethylenimine polymers exhibit effective transmembrane transportation of bio-macromolecule (such as large plasmids) into cells through the unique hydrophobic and lipophobic properties of fluorocarbon chains. Fluorinated polymers further enhance the nanoparticle delivery across the cell membrane and promote endosome/lysosome escape, which prevents enzymatic degradation of trapped polymer/nucleic acid complexes.

### Formulation, Optimization, Functionalization, and Characterization of the PDGFRB-Targeting, shTXND5 Plasmid-Encapsulated PEI Nanoparticles

A core-shell nanoparticle that displays peptides targeting PDGFRB while encapsulating a plasmid expressing short hairpin RNAs (shRNA) silencing TXNDC5 (FIG. 2A) was engineered. The ‘core’ of nanoparticles was the fluorinated polyethylenimine (PEI), which was used to condense plasmid DNA (FP/DNA), and the ‘shell’ was biocompatible hyaluronic acid (HA). HA was functionalized with PEG side chains and the PDGFRB-targeting peptide was anchored on the head of PEG. HA increased the circulating lifetime of the nanoparticle in vivo. The PDGFRB targeting nanoparticles were formed through electrostatic interaction by mixture of the negatively charged HA-functionalized PEG and positively charged FP/DNA complexes.

### Formulation and Optimization of the Plasmid-Encapsulated PEI Complex for Plasmid Endosome/Lysosome Escape

The formulation of the fluorinated polyethylenimine/plasmid (FP/DNA) core for its ability to drive an efficient plasmid endosome/lysosome escape and nucleus targeting in cells was optimized. A library of PEI core in which each polyethylenimine molecule contains 5, 6, 7, 8, 9, or 10 heptafluorobutyric acid groups (FIG. 2A), for the potency to deliver functional EGFP-expressing plasmids to cultured fibroblasts was tested. Fluorescence images detected the highest eGFP signaling in cells treated with the PEI core composed of the polyethylenimine molecule with 7 heptafluorobutyric acid groups (treatment of 24 hrs, data not shown). The PEI core was prioritized with 7 heptafluorobutyric acid groups for its superior potency of efficient endosomal/lysosome escape and nucleus targeting of functional plasmids.

### Functionalization and Characterization of the PDGFRB-Targeting, shTXNDC5 Plasmid-Encapsulated PEI Nanoparticles

The PEI/DNA core was functionalized with the shell composed the hyaluronic acid (HA)-PEG-PDGFRB targeting peptide molecules (FIG. 2A). HA functionalization increased the circulating lifetime of the nanoparticle in vivo. The PDGFRB-targeting peptide (CSRNLIDC (SEQ ID NO: 4)) was hypothesized to enhance the targeting and internalization of nanomaterials in activated fibroblasts in which the PDGFRB expression is markedly increased. Shell and functionalization increased the diameter of the PEI nanoparticle (measured by TEM and DLS, data not shown) and reduced the Zeta potential (data not shown).

### PDGFRB-Targeting NPs Effectively Deliver shTXNDC5 Plasmids to Activated Fibroblasts In Vitro

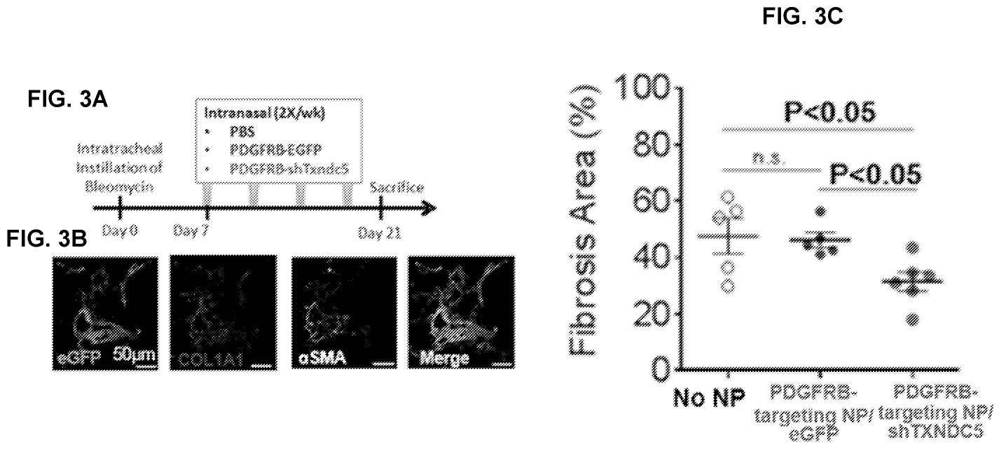

In vitro data in FIG. 2B demonstrated that this PDGFRB-targeting nanoparticle carrying shTXNDC5-expressing plasmids significantly reduced TXNDC5 expression in mouse fibroblasts when compared to PDGFRB-targeting nanoparticle carrying eGFP-expressing plasmids (treatment of 30 min, 500 ng plasmid in a 24-well). Non-targeting nanoparticles carrying shTXNDC5-expressing plasmids had limited effect on TXNDC5 expression (data not shown). Moreover, preliminary in vivo data showed four intranasal injections of the PDGFRB-targeting nanoparticles encapsulating shTXNDC5 plasmids reduced bleomycin-induced lung fibrosis, when compared to mice subjected to PDGFRB-targeting nanoparticles encapsulating control eGFP plasmids (FIG. 3C). A liposome displaying a non-functional scrambled PDGFRB-targeting peptide (CTSDHAVC (SEQ ID NO: 5)) which served as an additional control in Examples 1 and 2 was engineered, which test the therapeutic effectiveness of PDGFRB-targeting nanoparticles to deliver TXNDC5 shRNA and treat pulmonary fibrosis induced by bleomycin using mouse models (data not shown).

### Example 1: Determining the Therapeutic Effectiveness of PDGFRB-Targeting Nanoparticles for Delivering shTXNDC5 Plasmids to Fibroblasts and Treating Pulmonary Fibrosis (PF) Induced by Bleomycin

### Introduction

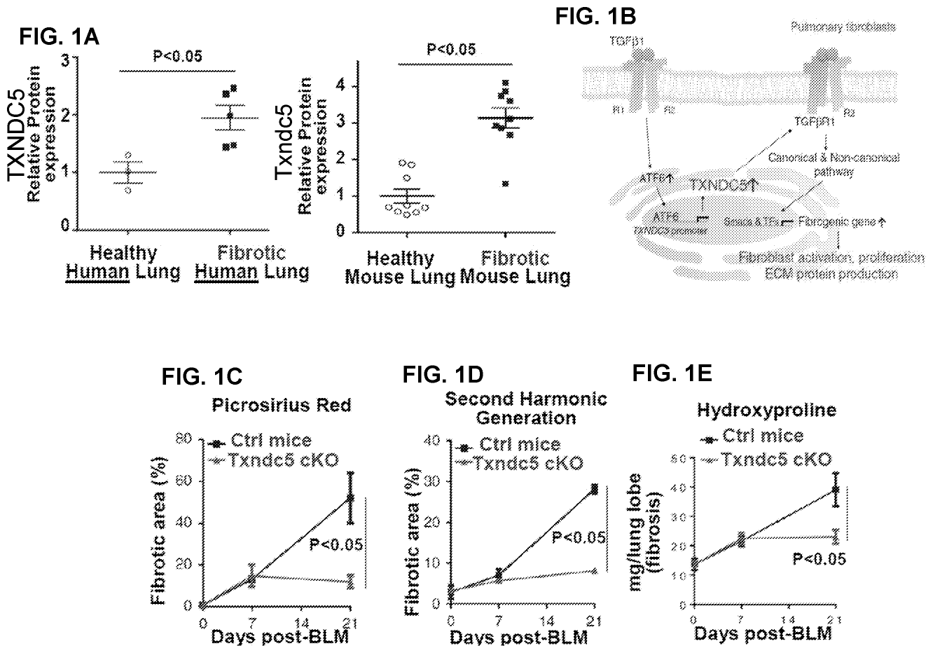

The overall goal was to determine the therapeutic effect of PDGFRB-targeting, TXNDC5 shRNA expressing plasmid-encapsulated nanoparticles in reducing pulmonary fibrosis. Bleomycin is an FDA-approved anti-cancer drug but causes pulmonary fibrosis in patients. This led to the well-established, bleomycin-induced PF mouse model which has successfully prompted new drug developments shown in Wollin et al. (J. Pharmacol. Exp., 2014, 349(2), 209-220) and Oku et al. (Eur. J. Pharmacol., 2008, 590(1-3), 400-408). Previous studies demonstrated that fibroblast TXNDC5 is significantly induced in a bleomycin-induced mouse PF model, and also in fibrotic human lung autopsy samples (FIG. 1A). In addition, genetic deletion of TXNDC5 in fibroblasts significantly reduced bleomycin-induced PF in mice (FIGS. 1C-1E). These data provide a mechanistic basis for targeting fibroblast TXNDC5 to treat pulmonary fibrosis; nevertheless, the challenge is to develop an effective strategy to deliver therapeutics inhibiting TXNDC5 in activated fibroblasts. To overcome this, a nanoparticle which effectively delivers TXNDC5-targeting shRNA to activated fibroblasts via a targeting peptide against Platelet Derived Growth Factor Receptor Beta (PDGFRB) was engineered. Single-cell profiling and mechanistic studies have demonstrated that PDGFRB expression is significantly increased in activated fibroblasts in fibrotic lungs. A novel nanoparticle which displays a PDGFRB-targeting peptide and encapsulates plasmids expressing short hairpin RNA (shRNA) against TXNDC5 (FIG. 2) was engineered. The therapeutic effectiveness of this nanomedicine approach in treating pulmonary fibrosis was tested in a mouse model subjected to bleomycin.

### PDGFRB-Targeting, shTXNDC5 Expressing Plasmid-Encapsulated Nanoparticles

Formulation of the PDGFRB-targeting nanoparticle was described above in FIG. 2A. The size and zeta potential are determined by dynamic light scattering (DLS) and transmission electron microscopy (TEM).

### Application of PDGFRB-Targeting Nanoparticles in Treating Pulmonary Fibrosis in Mice Subjected to Bleomycin

The therapeutic effectiveness of this nanomedicine approach in treating pulmonary fibrosis was tested in a mouse model subjected to bleomycin. Procedures of bleomycin-induced PF in mice have been described in a previous study by Lee et al. (Nat. Commun., 2020, 11(1), 4254). Briefly, C57BL/6J mice are intratracheally (IT) instilled with bleomycin (3 mg/kg) or PBS control. 7 days after IT injection, mice were intranasally (IN) instilled with PDGFRB-targeting shTXNDC5 nanoparticles (6 μg plasmid/per mice, in 20 μL solution) twice a week for 2 more weeks. A total of 5 groups were employed in this study, following the scheme depicted in FIG. 3A. 6-8 week-old male mice were used for the following groups: 1) PBS IT instilled baseline control, PBS IN, 2) bleomycin IT instilled, non-targeting nanoparticles instilled/eGFP control plasmids IN, 3) bleomycin IT instilled, PDGFRB-targeting nanoparticles instilled/eGFP control plasmids IN, 4) bleomycin IT instilled, PDGFRB-targeting nanoparticles instilled/shTXNDC5-expressing plasmids IN, and 5) bleomycin IT instilled, non-functional scrambled PDGFRβ-targeting peptide (CTSDHAVC (SEQ ID NO: 5)) nanoparticles instilled/shTXNDC5-expressing plasmids IN. On Day 0, 7, 14, and 21, pressure volume loops, lung compliance, and resistance was measured on a FlexiVent system. At 21 days, animals were sacrificed by exsanguination under anesthesia. A portion of the lungs were then rapidly digested into single-cell suspension for single cell RNA sequencing. Lungs were also divided for histology (fixed in 10% buffered formalin), RNA, and protein analyses. Histology with picrosirius red (for collagen) and immunofluorescence was performed to co-localize active fibrosis (αSMA, Eln1, Col1a1) with fibroblasts. Fixed lungs were also analyzed for hydroxyproline content (collagen production) and second harmonic generation (SHG, collagen imaging). Expression of αSMA, ELN1, COL1A1, and TXNDC5 will be measured by RT-PCR and Western blots. Bronchoalveolar lavage (BAL) was performed by injecting 0.5 mL of PBS into the lung and gently aspirating the fluid three times prior to lung harvest. Lavage protein and cell count were performed to assess for the effect of nanoparticles on lung inflammation. The same experiments were conducted and repeated in female mice. Gender differentiation is mandatory as female mice are less prone to develop fibrosis after bleomycin.

### Bio-Distribution of PDGFRB-Targeting Nanoparticles In Vivo

To visually determine the functional distribution of this PDGFRB-targeting nanoparticle in vivo, both the nanoparticle and overexpressed gene were tracked. Nanoparticles can be imaged by labeling the PEG with Cyanine 5. eGFP-expressing plasmids can be encapsulated in the targeted nanoparticles to determine the bio-distribution of functional plasmids. Organ fluorescence imaging (IVIS 200 Imaging System, PerkinElmer) was performed, as well as histological and immunohistochemical staining of cross-sections of tissues to systematically determine the bio-distribution of the PEG and eGFP.

### Tolerable doses, Toxicity, and Immunogenicity

Mice were subjected to 0.5, 6, 20, and 50 μg shTXNDC5 plasmid/per mice to determine the tolerable doses. Liver function was assessed by the activities of alanine aminotransferase (ALT), alkaline phosphatase (ALP), and aspartate aminotransferase (AST). Spleen was isolated for flow cytometry analyses for immune cells (macrophages, T cells, and B cells) and their state of activation. In addition, amylase, urea nitrogen, calcium, cholesterol, glucose, total bilirubin, and total proteins were determined by a Vet Axcel blood chemistry analyzer (Alfa Wasserman). Lungs, heart, liver, spleen, and kidney were collected from the same mice for H&E staining and histology examination. Detailed protocols are listed in previous studies.

### Results

Strongly supported by the data using both global and fibroblast specific knockdown of TXNDC5 (FIGS. 1C-1E) in bleomycin-induced PF mouse models demonstrated by Lee et al. (Nat. Commun., 2020, 11(1), 4254), PDGFRB-targeting nanoparticle reduced fibroblast TXNDC5, and therefore fibrosis in mice induced by bleomycin was treated. This was supported by the preliminary in vivo results showing this PDGFRB-targeting nanoparticle effectively delivered functional plasmids to activated fibroblasts (FIG. 2B). Collagen type I alpha 1 chain (COL1A1) and α-SMA are markers of fibroblast activation. Preliminary in vivo data demonstrated that this targeted nanomedicine approach (four intranasal injections of 6 μg plasmid/per mice, FIG. 3A) delivers functional eGFP plasmids to COL1A1- and α-SMA-expressing cells (FIG. 3B). Moreover, preliminary results showed a significantly reduction of fibrotic area in bleomycin-treated mouse lung when shTXNDC5-expressing plasmids were delivered by the PDGFRB-targeting nanoparticle (FIG. 3C).

### Example 2: PDGFRB-Targeting Polyelectrolyte Complex Micelles Successfully Deliver TXNDC5-Targeting CRISPR Plasmids to Activated Fibroblasts, Leading to the TXNDC5 Reduction

The overall goal was to determine the therapeutic effect of PDGFRB-targeting, TXNDC5 CRISPR expressing plasmid-encapsulated nanoparticles in reducing pulmonary fibrosis. A formulation of the PDGFRB-targeting polyelectrolyte complex micelle composed of PEG-polyarginine polymers and a TXNDC5-targeting CRISPR-Cas9 vector was generated (FIG. 4). Briefly, the dried targeting peptide-poly(ethylene glycol)-poly(L-arginine) was dissolved in Milli-Q purified water to 500 ng/μL. The prepared therapeutic plasmid was aliquoted to 500 ng/μL. In a 2 mL microcentrifuge tube, 100 μL of Milli-Q purified water was added followed by the addition of 2 μL of the polycation solution. This was pipetted up and down at least 5 times. For the 1:2 w/w ratio between polycation and plasmid, 1 μL of the plasmid solution was then added and the overall solution gently pipetted 10 times, avoiding the formation of droplets at the side of the tube. The microcentrifuge tube was placed over ice for approximately 20 minutes. 100 μL of Milli-Q purified water was added prior analysis by dynamic light scattering or nanoparticle tracking analysis.

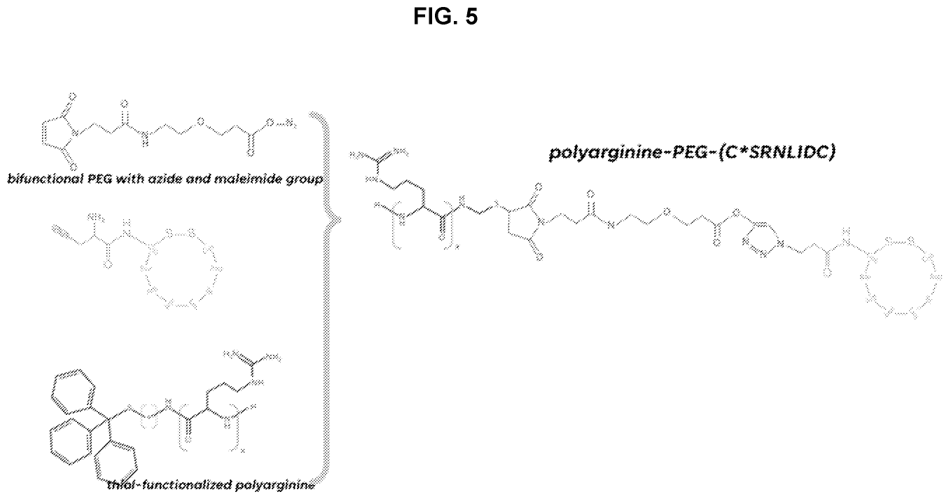

The polycation component of the polyelectrolyte complex micelle (PCM), targeting Peptide(C*SRNLIDC (SEQ ID NO: 4) shown in for PDGFRB targeting, which can be changed to other targeting peptides; see FIG. 5). Polyethylene Glycol-Poly-L-Arginine, was synthesized through first “clicking” the azide group of a heterobifunctionalized poly(ethylene glycol) at the alkyne end group functionalized targeting peptide via cycloaddition. The other end of the heterobifunctionalized poly(ethylene glycol), a maleimide group, was then “clicked” to the thiol-functionalized end of the poly(L-arginine) via Michael addition. Thiol functionalized poly(L-arginine) was purchased from Alamanda Polymers (x=30, MW=5,800 Da).

This poly(L-arginine) was trityl protected on the C-terminal which needs to first be cleaved prior to use. 100 mg of the trityl protected thiol-functionalized poly(L-arginine) was dissolved in 2 mL of a mixture of trifluoroacetic acid (TFA)/triethylsilane (Et3 SiH) (98:2 v/v) under inert gas and stirred at room temperature for 2 hours. This mixture was precipitated in a 10-fold excess of diethyl ether (Et2O) and recovered via filtration. The thiol-functionalized poly(L-arginine) was then frozen and dried to powder under lyophilization (Labconco).

Poly(ethylene glycol) functionalized with both an azide group and a maleimide group was purchased from NanoSoft Polymer (MW=2000 Da). The alkyne end group functionalized targeting peptide was purchased from GenScript (Purity: ≥95%, Length: 8, Disulfide Bridge: 1-8). These components are connected by the copper-catalyzed azide-alkyne cycloaddition reaction. Ascorbate was used as a reducing agent. Approximately 16 mg of the alkyne-functionalized peptide is added to a glass vial with a magnetic stir bar in 1 mL of Milli-Q purified water. 35 mg (2.5-fold excess to the alkyne group) of the azide-functionalized poly(ethylene glycol) itself dissolved in 500 μL Milli-Q purified water was then added followed by 400 μL of 7 mg/mL CuSO₄. 175 mg of sodium ascorbate in 1 mL of Milli-Q purified water is then added. The vial is purged of oxygen with nitrogen and capped. The reaction solution is allowed to react for 2 hours under stirring.

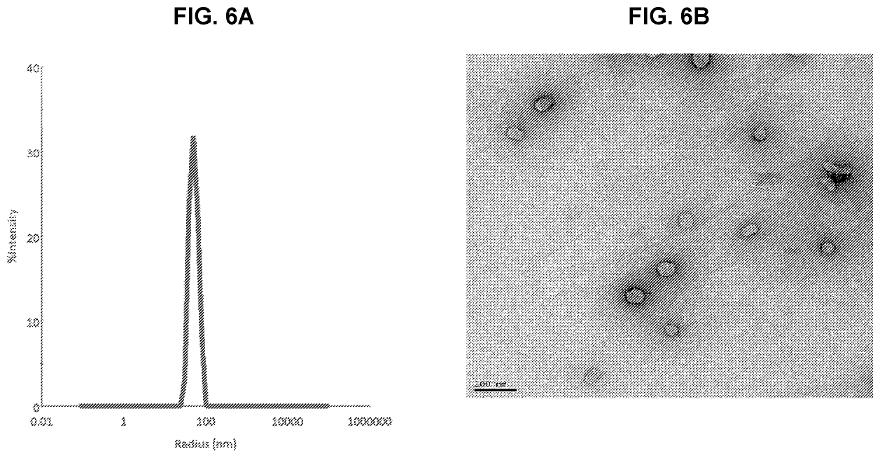

The radius of plasmid-encapsulated, PDGFRB-targeting polyelectrolyte complex micelles is ˜20-100 nm, as demonstrated by (FIG. 6A) dynamic light scattering (DLS) and (FIG. 6B) negatively stained transmission electron microscopy (TEM). 100 mg of thiol-functionalized poly(l-arginine) was dissolved in 200 μL of Milli-Q purified water and sonicated for 10 minutes. This solution was then added to the maleimide-functionalized poly(ethylene glycol) targeting peptide solution along with 10 mg of TCEP (tris(2-carboxyethyl)phosphine). This reaction solution was again purged with nitrogen and then allowed to react 48 hours. The formed poly(L-arginine)-poly(ethylene glycol)-targeting peptide solution was dialyzed against Milli-Q purified water (MWCO=2 k) for 48 hours with water replacement every 15 minutes for the first 2 hours and then frequent replacements over the following hours. The purified solution is then frozen and lyophilized for 48 hours.

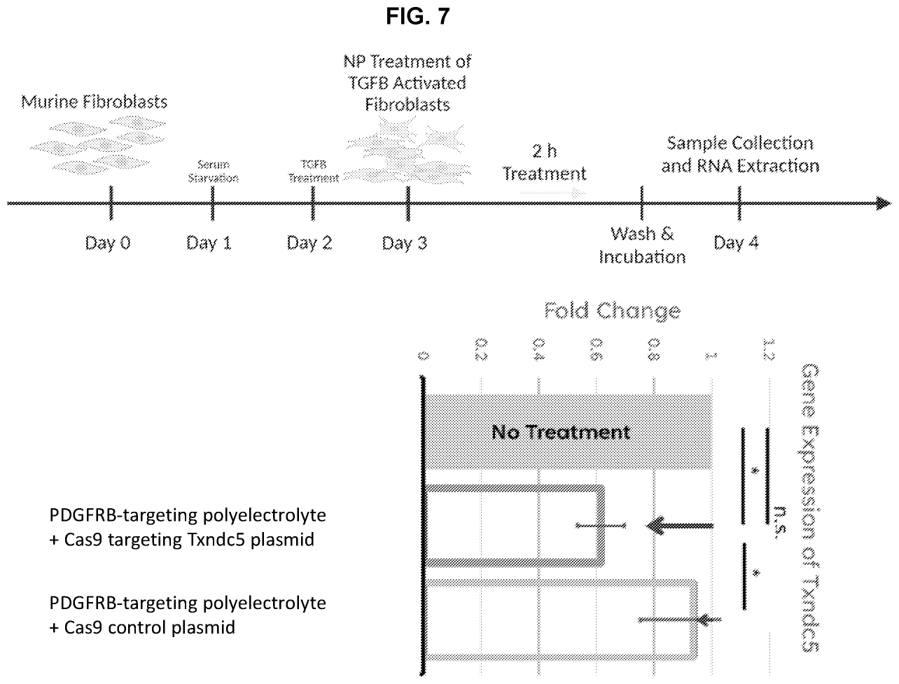

The PDGFRB-targeting polyethylene-glycol-poly-L-arginine polyelectrolyte complex micelles encapsulating Txndc5-targeting CRISPR-Cas9 plasmids as described above successfully suppress Txndc5 expression in mouse fibroblasts. Mouse fibroblasts were first activated by TGF-β and then treated with the polyelectrolyte complex micelles which encapsulate Txndc5-targeting CRISPR-Cas9 plasmids or control plasmids (FIG. 7). The Txndc5-targeting CRISPR-Cas9 vector was a plasmid engineered to express Cas9 driven by a mouse collal promoter that is preferentially activated in fibroblasts. The Txndc5-targeting CRISPR-Cas9 vector also contains two U6 promoter-driven single-guide RNAs (sgRNAs) targeting introns 1 and 3 of Txndc5. The control plasmid was engineered to express Cas9 driven by a mouse col1a1 promoter and contains non-targeting guide RNAs. Decreased expression of Txndc5 mRNA in mouse fibroblasts treated with PDGFRB-targeting polyelectrolyte complex micelles encapsulating Txndc5-targeting Cas9 plasmids when compared to fibroblasts treated with PDGFRB-targeting polyelectrolyte complex micelles encapsulating control Cas9 plasmids.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

### Sequences

| Sequence | SEQ ID NO: |
|---|---|
| VHPKQHR | 1 |
| QAHPHVD | 2 |
| DITWDQLWDLMK | 3 |
| CSRNLIDC | 4 |
| CTSDHAVC | 5 |

What is claimed is:

1. A targeted nanoparticle, comprising an inhibitor of thioredoxin domain-containing 5 (TXNDC5).

2. The targeted nanoparticle of claim 1, wherein the targeted nanoparticle comprises a polyethylene glycol 2000 (PEG) domain, and a Platelet Derived Growth Factor Receptor Beta (PDGFRB) targeting molecule.

3. The targeted nanoparticle of claim 2 further comprising poly-L-arginine, hyaluronic acid (HA), and/or a fluorinated polyethylenimine (PEI).

4. The targeted nanoparticle of any one of claims 1-3, wherein the PDGFRB targeting molecule comprises a peptide comprising the amino acid sequence CSRNLIDC (SEQ ID NO: 4).

5. The targeted nanoparticle of claim 4, wherein the PDGFRB targeted nanoparticle comprises CSRNLIDC (SEQ ID NO: 4), the PEG domain, and poly-L-arginine.

6. The targeted nanoparticle of claim 4, wherein the PDGFRB targeted nanoparticle comprises CSRNLIDC (SEQ ID NO: 4), the PEG domain, fluorinated polyethylenimine (PEI), and HA.

7. The targeted nanoparticle of any one of claims 2-6, wherein the inhibitor of TXNDC5 is a short hairpin RNA (shRNA) silencing TXNDC5 (shTXNDC5), a TXNDC5-targeting small interfering (siRNA), and/or a TXNDC5-targeting CRISPR plasmid.

8. The targeted nanoparticle of any one of claims 2-7, wherein the inhibitor of TXNDC5 comprises a concentration of about 2 μM.

9. The targeted nanoparticle of any one of claim 2-8, wherein the PEG domain comprises PEG having an average molecular weight of about 1,000 to about 100,000 Daltons.

10. A pharmaceutical composition, comprising:
a therapeutically effective amount of the targeted nanoparticle of any one of claims 1-9; and
a pharmaceutically acceptable carrier, solvent, adjuvant, and/or diluent.

11. The pharmaceutical composition of claim 10, wherein the pharmaceutical composition is formulated for oral, intravenous, topical, ocular, buccal, systemic, nasal, tracheal, injection, transdermal, rectal, or vaginal administration.

12. The pharmaceutical composition of claim 10, wherein the pharmaceutical composition is formulated for inhalation or insufflation.

13. A pharmaceutical composition for pulmonary delivery of an inhibitor of TXNDC5, comprising:
a) a targeted nanoparticle comprising
i) a PDGFRB targeting molecule;
ii) a polyethylene glycol (PEG) domain; and
iii) the inhibitor of TXNDC5;
b) pharmaceutically acceptable carrier,
wherein the composition is formulated such that once administered to the lung, it results in the delivery of the inhibitor of TXNDC5 to a lung cell.

14. The pharmaceutical composition of claim 13 further comprising poly-L-arginine, hyaluronic acid (HA), and/or a fluorinated polyethylenimine (PEI).

15. The pharmaceutical composition of either claim 13 or claim 14, wherein the PDGFRB targeting molecule comprises a peptide comprising the amino acid sequence CSRNLIDC (SEQ ID NO: 4).

16. The pharmaceutical composition of claim 15, wherein the PDGFRB targeted nanoparticle comprises CSRNLIDC (SEQ ID NO: 4), the PEG domain, and poly-L-arginine.

17. The pharmaceutical composition of claim 15, wherein the PDGFRB targeted nanoparticle comprises CSRNLIDC (SEQ ID NO: 4), the PEG domain, fluorinated polyethylenimine (PEI), and HA.

18. The pharmaceutical composition of any one of claims 13-17, wherein the inhibitor of TXNDC5 is a short hairpin RNA (shRNA) silencing TXNDC5 (shTXNDC5), a TXNDC5-targeting small interfering (siRNA), and/or a TXNDC5-targeting CRISPR plasmid.

19. The pharmaceutical composition of any one of claim 13-18, wherein the PEG domain comprises PEG having an average molecular weight of about 1,000 to about 100,000 Daltons.

20. A method of treating a lung disorder in a subject, comprising:
administering to the subject a therapeutically effective amount of the pharmaceutical composition of any one of claims 10-19 comprising a targeted nanoparticle comprising an inhibitor of TXNDC5, wherein the targeted nanoparticle is preferentially targeted to fibrotic activated fibroblast cells associated with the lung disorder; and
reducing fibrosis at the site of the fibrotic activated fibroblast cells.

21. The method of claim 20, wherein the lung disorder is pulmonary fibrosis.

22. The method of claim 21, wherein the pulmonary fibrosis is an idiopathic pulmonary fibrosis.

23. A method of promoting fibroblast wound healing in a subject, comprising:
administering to the subject a therapeutically effective amount of the pharmaceutical composition of any one of claims 10-19 comprising a targeted nanoparticle comprising an inhibitor of TXNDC5, wherein the targeted nanoparticle is preferentially targeted to fibrotic fibroblast cells associated with the fibroblast wound; and
reducing fibrosis at the site of the fibrotic fibroblast cells.

24. The method of any one of claims 20-23, wherein the probability of survival of the individual is at least about 10% greater than an expected probability of survival without administration of the pharmaceutical composition.
